(19) United States
(12) Patent Application Publication
(10) Pub. No.: US 2026/0209662 A1
(43) Pub. Date: Jul. 23, 2026

(54) Device And Method For Cellular Arrangement And Microfluidic Patterning

(71) Applicant: Wisconsin Alumni Research Foundation, Madison, WI (US)

(72) Inventors: Jose Ayuso, Verona, WI (US); Rithvik Turaga, Madison, WI (US); Seth Zima, Madison, WI (US)

(21) Appl. No.: 19/444,885

(22) Filed: Jan. 9, 2026

Related U.S. Application Data
(60) Provisional application No. 63/746,657, filed on Jan. 17, 2025.

(51) Int. Cl. C12M 1/32; C12M 1/00; C12M 1/12; C12M 1/34; C12M 3/06

(52) U.S. Cl. CPC C12M 23/12; C12M 23/16; C12M 25/14; C12M 29/00; C12M 41/36

(57) ABSTRACT

A cellular arrangement and microfluidic patterning device and a method of studying the positional context of cells are provided. The device includes a plate having a first face with a well formed therein. The well is partially defined by a bottom surface spaced from the first face. A cavity is partially defined by a first surface directed away from the well and a second surface spaced from the first surface and directed towards the well. A voxel, aligned with the well, projects from the first surface into the cavity and terminates at a terminal surface spaced from the second surface. An aperture extends between the bottom surface of the well and the terminal surface of the voxel.

### CROSS REFERENCE TO RELATED APPLICATION

This Application claims priority of U.S. Provisional Patent Application No. 63/746,657 filed Jan. 17, 2025, the entirety of which is incorporated herein by reference.

### FIELD OF THE INVENTION

This invention relates generally to spatial biology, and in particular, to a method and a device for cellular arrangement and microfluidic patterning which provide a non-destructive tool to study cells in a spatial manner and which allow for the concise manipulation of nutrients, metabolites, drugs, and the like to be delivered to the cells.

### BACKGROUND AND SUMMARY OF THE INVENTION

Spatial biology is directed to the study of how biological molecules, cells and tissue are organized, interact, and function in their natural two-dimensional or three-dimensional environment. More specifically, spatial biology involves the use of various techniques to allow researchers to study the positional context of molecules and cells in a tissue sample. Using these spatial biology techniques, researchers can visualize the spatial architecture of molecules, cells and tissues and how the molecules, cells and tissues interact and relate to each other. It can be appreciated that the information obtained utilizing spatial biology techniques may help researchers develop new strategies for preventing or treating diseases, such as cancer, infection, neurological conditions, and metabolic disorders.

While the use of spatial biology has exhibited enormous potential for researchers, current techniques have certain limitations. For example, current spatial biology technologies generate a large amount of complex data, which can be difficult to analyze and require a significant amount of data storage. Further, analyzing multiple labels and thousands of data points can be time-consuming. In addition, the tissue specimens used in spatial biology can be difficult to preserve and require destruction of the sample due to the pre-processing steps. In view of the foregoing, it can be appreciated that current techniques to study spatial biology in vitro are limited by cost.

Therefore, it is a primary object and feature of the present invention to provide a device for cellular arrangement and microfluidic patterning which allows for the generation of intricate cell culture systems and the non-invasive spatial analysis thereof.

It is a further object and feature of the present invention to provide a device for cellular arrangement and microfluidic patterning which allows for real-time investigation into the spatiotemporal evolution of a cell culture system.

It is a still further object and feature of the present invention to provide a device for cellular arrangement and microfluidic patterning which alleviates the need for researchers to design and manufacture specialized microfluidic devices for each project.

It is a still further object and feature of the present invention to provide a device for cellular arrangement and microfluidic patterning which is simple to manufacture and inexpensive to utilize.

In accordance with the present invention, a cellular arrangement and microfluidic patterning device is provided. The cellular arrangement and microfluidic patterning device includes a plate with a first face having a well formed therein. The well is partially defined by a bottom surface spaced from the first face. A cavity is partially defined by a first surface directed away from the well and a second surface spaced from the first surface and directed towards the well. A voxel projects from the first surface into the cavity and terminates at a terminal surface spaced from the second surface. The voxel is axially aligned with the well. An aperture extends between the bottom surface of the well and the terminal surface of the voxel.

The terminal surface of the voxel is defined by a generally rectangular peripheral edge. The first surface is spaced from the second surface by a distance. The terminal surface of the voxel is spaced from the second surface by a distance which is less than the distance between the first and second surfaces. The well may be a first well and the voxel may a first voxel. As such, the plate may include a second well formed in the first face. The second well is partially defined by a bottom surface spaced from the first face. A second voxel projects from the first surface into the cavity and terminates at a terminal surface spaced from the second surface. The second voxel is axially aligned with the second well. An aperture extends between the bottom surface of the second well and the terminal surface of the second voxel.

The terminal surface of the second voxel is defined by a generally rectangular peripheral edge. The terminal surfaces of the first and second voxels lie in a common plane. The first and second voxels are spaced from each other along the first surface by a distance. A media transfer tool may also be provided. The media transfer tool includes a panel and first and second tubular projections extending from the panel. The terminal ends of the projections are configured to be received in corresponding first and second wells of the plate. A guide may be receivable on the first face of the plate. The guide includes a panel having first and second faces and first and second apertures therethrough that are alignable with corresponding first and second wells in the plate. Indicia are provided on the first face to facilitate identification of locations of first and second wells in the plate.

In accordance with a further aspect of the present invention, a cellular arrangement and microfluidic patterning device is provided. The cellular arrangement and microfluidic patterning device includes a plate having a generally planar face and an array of wells provided in the face. Each well of the array of wells is partially defined by a bottom surface spaced from the face. A cavity is partially defined by a first surface directed away from the array of wells and a second surface spaced from the first surface and is directed towards the array of wells. A plurality of voxels project from the first surface into the cavity. Each voxel of the plurality of voxel terminates at a terminal surface spaced from the second surface and is axially aligned with a corresponding well of the array of well. The plate further includes a plurality of apertures. Each aperture extends between the bottom surface of one of the wells of the array of wells and the terminal surface of a corresponding voxel of the plurality of voxels.

Each terminal surface of the plurality of voxels is defined by a generally rectangular peripheral edge. At least one voxel of the plurality of voxels has a rectangular cross-section. The terminal surfaces of the plurality of voxels lie in a common plane. The first surface is spaced from the second surface by a distance. The terminal surfaces of the plurality of voxels are spaced from the second surface by a distance which is less than the distance between the first and second surfaces. It is contemplated for each voxel of the plurality of voxels to have identical configurations. Each voxel of the plurality of voxels is spaced from an adjacent voxel of the plurality of voxels by a distance.

A media transfer tool may also be provided. The media transfer tool includes a panel and an array of tubular projections extending from the panel and having terminal ends. The terminal ends of the projections are receivable in a corresponding well of the array of wells of the plate. The array of wells may be arranged in rows and columns in the face of the panel. A guide is receivable on the first face of the plate. The guide includes a panel having first and second faces and an array of apertures there between. Each aperture of the array of apertures is alignable with corresponding well of the array of wells. First and second sets of indicia are provided along the first face of the panel. The first set of indicia extends along a first axis and identifies each row of wells of the array of wells. The second set if indicia extends along a second axis and identifies each column of wells of the array of wells.

A method of studying the positional context of cells is provided. The method includes the step of providing an array of wells in a face of a plate. The plate includes a cavity communicating with each of the wells of the array of wells and a plurality of voxels projecting into the cavity. Each voxel is aligned with a corresponding well and from a first surface into the cavity. Each voxel of the plurality of voxel terminates at a terminal surface. Three-dimensional hydrogel figures are deposited on at least a portion of the terminal surfaces of the plurality of voxels. Media is deposited media in the cavity such that the media surrounds the hydrogel figures.

Each hydrogel figure includes cells suspended therein. The arrays of wells may be arranged in rows and columns. Indicia on the face of the plate may be provided to identify the rows and columns of the array of wells. The hydrogel figures deposited on the at least the portion of the terminal surfaces of the plurality of voxels may be pinned thereon. The array of wells may be with the media and the media may be selectively drawn out of the array of wells with a media transfer tool. The media transfer tool includes a panel and an array of tubular projections extending from the panel and having terminal ends. The terminal ends of the projections are receivable in corresponding wells of the array of wells of the plate.

### BRIEF DESCRIPTION OF THE DRAWINGS

Preferred exemplary embodiments of the invention are illustrated in the accompanying drawings in which like reference numerals represent like parts throughout, and in which:

FIG. 1 is an isometric view of a cellular arrangement and microfluidic patterning device in accordance with the present invention;

FIG. 2 is a side elevational view of the cellular arrangement and microfluidic patterning device of FIG. 1;

FIG. 3 is a cross sectional view of the cellular arrangement and microfluidic patterning device of the present invention taken along line 3-3 of FIG. 1;

FIG. 4 is a cross sectional view of the cellular arrangement and microfluidic patterning device of the present invention taken along line 4-4 of FIG. 2;

FIG. 5 is an enlarged, top plan view of a well of the cellular arrangement and microfluidic patterning device of FIG. 3;

FIG. 6 is an enlarged, bottom plan view of a voxel of the cellular arrangement and microfluidic patterning device of FIG. 3;

FIG. 7 is a cross sectional view, similar to FIG. 3, show a first step in the loading of the cellular arrangement and microfluidic patterning device of the present invention;

FIG. 8 is a cross sectional view, similar to FIG. 3, show a further step in the loading of the cellular arrangement and microfluidic patterning device of the present invention;

FIG. 9 is a cross sectional, isometric view of first and second wells of the cellular arrangement and microfluidic patterning device of the present invention;

FIG. 10 is a cross sectional view of first and second wells of the cellular arrangement and microfluidic patterning device of the present invention loaded with first and second hydrogels;

FIG. 11 is an isometric view of a sample transport device for use in conjunction with the cellular arrangement and microfluidic patterning device of the present invention;

FIG. 12 is an exploded, isometric view of the sample transport device of FIG. 11 and the cellular arrangement and microfluidic patterning device of the present invention;

FIG. 13 is an isometric view of the sample transport device received in the cellular arrangement and microfluidic patterning device of the present invention;

FIG. 14 is a top plan view of an indicia plate positioned on the cellular arrangement and microfluidic patterning device of the present invention;

FIG. 15 is cross sectional view of the indicia plate positioned on the cellular arrangement and microfluidic patterning device taken along line 15-15 of FIG. 14; and

FIG. 16 is cross sectional view of the indicia plate positioned on the cellular arrangement and microfluidic patterning device taken along line 16-16 of FIG. 14.

### DETAILED DESCRIPTION OF THE DRAWINGS

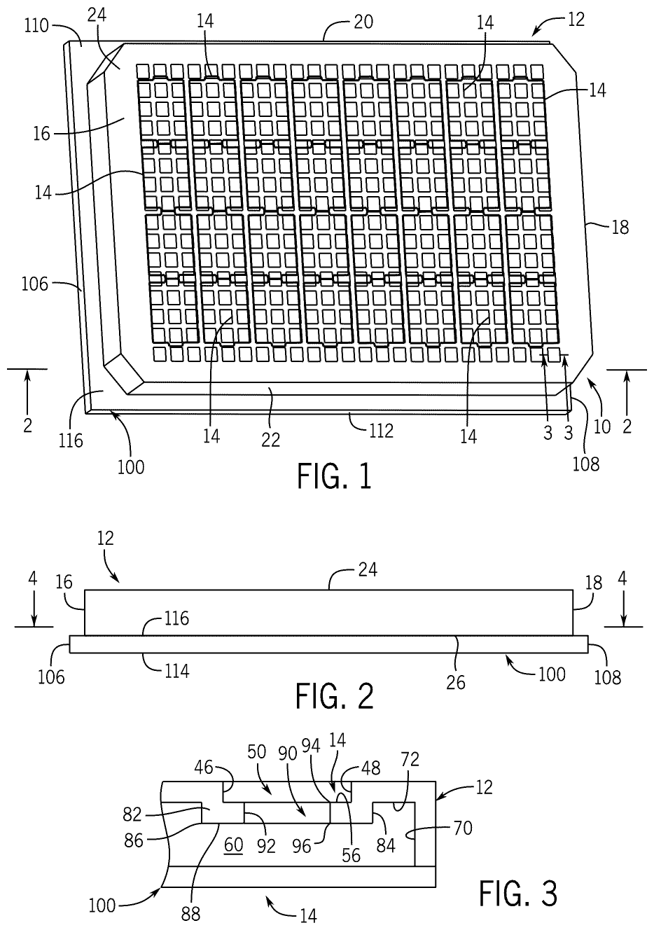

Referring to FIGS. 1-2, a cellular arrangement and microfluidic patterning device in accordance with the present invention is generally designated by the reference numeral 10. Device 10 includes well plate 12 fabricated from a plastic material such as polyvinylchloride, polystyrene or polypropylene and has a generally rectangular configuration. However, other configurations are possible without deviating from the scope of the present invention.

Well plate 12 is defined by first and second generally parallel ends 16 and 18, respectively, interconnected first and second generally parallel sidewalls 20 and 22, respectively, perpendicular thereto. Well plate 10 further includes upper surface 24 and lower surface 26. A plurality of wells, designated collectively by the reference numeral 14, are provided in upper surface 24 of well plate 12. By way of example, in the depicted embodiment, well plate 12 includes three hundred eighty four (384) individual wells 14 arranged in sixteen (16) rows and twenty-four (24) columns. However, it can be understood that the number and arrangement of wells 14 in well plate 12 may be varied, without deviating from the scope of the present invention.

Each well 14 in upper surface 24 of well plate 12 is identical in structure. As such, the description hereinafter of well 14 located at the intersection of row 16 and column 24, FIG. 1, is understood to described each of the plurality of wells 14, as if fully described herein. Referring to FIGS. 3, 5 and 7-8, well 14 is defined first and second generally parallel sidewalls 34 and 36, respectively, generally parallel to first and second sides 20 and 22, respectively, of well plate 12 and generally perpendicular to first and second ends 16 and 18, respectively, of well plate 12 and by third and fourth generally parallel sidewalls 38 and 40, respectively, generally perpendicular to first and second sides 20 and 22, respectively, of well plate 12 and generally parallel to first and second ends 16 and 18, respectively, of well plate 12. First face 42 of first sidewall 34, first face 44 of second sidewall 36, first face 46 of third sidewall 38 and first face 48 of fourth sidewall 40 are directed toward interior 50 of well 14. Lower edges of first face 42 of first sidewall 34, first face 44 of second sidewall 36, first face 46 of third sidewall 38 and first face 48 of fourth sidewall 40 are interconnected by lower face 56.

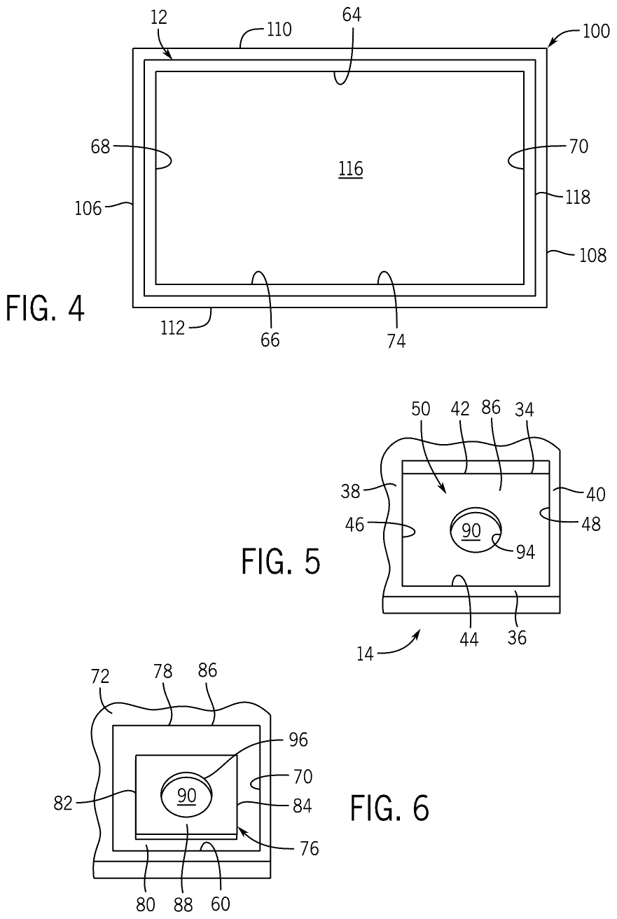

Referring to FIGS. 3-4 and 6, media cavity 60 is provided in lower surface 26 of well plate 12. Media cavity 60 is defined first and second generally parallel sidewalls 64 and 66, respectively, generally parallel to first and second sides 20 and 22, respectively, of well plate 12 and generally perpendicular to first and second ends 16 and 18, respectively, of well plate 12 and by third and fourth generally parallel sidewalls 68 and 70, respectively, generally perpendicular to first and second sides 20 and 22, respectively, of well plate 12 and generally parallel to first and second ends 16 and 18, respectively, of well plate 12. First, second, third and fourth sidewalls 64, 66, 68 and 70, respectively, are directed toward the interior of media cavity 60. The upper edges of first, second, third and fourth sidewalls 64, 66, 68 and 70, respectively, are interconnected by upper face 72. The lower edges first, second, third and fourth sidewalls 64, 66, 68 and 70, respectively, define opening 74 in lower surface 26 of well plate 12.

A plurality of voxels 76 depend from upper face 72 of well plate 12. Each voxel 76 is axially aligned with a corresponding well 14 in upper surface 24 of well plate 12 and has a generally square configuration. However, other configurations are possible without deviating from the scope of the present invention. In the depicted embodiment, each voxel is defined by first and second generally parallel sides 78 and 80, respectively, generally parallel to first and second sides 20 and 22, respectively, of well plate 12 and generally perpendicular to first and second ends 16 and 18, respectively, of well plate 12 and by third and fourth generally parallel sides 82 and 84, respectively, generally perpendicular to first and second sides 20 and 22, respectively, of well plate 12 and generally parallel to first and second ends 16 and 18, respectively, of well plate 12. The upper edges of first, second, third and fourth sides 78, 80, 82 and 84, respectively, intersect upper face 72. The lower edges of first, second, third and fourth sides 78, 80, 82 and 84, respectively, define pinning edge 86 extending about the outer periphery of terminal face 88. Terminal face 88 is generally planar and the terminal faces 86 of the plurality of voxels 76 lie in a common plane which is generally parallel to upper surface 24 and upper face 72. Passages 90 extend through the plurality of voxels 76 and are defined by a cylindrical inner surface 92 having a first lower edge 96 intersecting terminal face 56 of a corresponding voxel 76 so as to define an opening therein in communication with passage 90 and an upper edge 94 intersecting lower face 56 of a corresponding well 14 so as to define an opening therein in communication with passage 90.

Lid plate 100 is bonded to lower surface 26 of well plate 12. More specifically, lid plate 100 is fabricated from a plastic material such as polyvinylchloride, polystyrene or polypropylene and has a generally rectangular configuration. However, other configurations are possible without deviating from the scope of the present invention. Lid plate 100 is defined by first and second generally parallel ends 106 and 108, respectively, interconnected first and second generally parallel sidewalls 110 and 112, respectively, perpendicular thereto. Lid plate 100 further includes lower surface 114 and upper surface 116 having an outer peripheral portion 118 bonded to lower surface 26 of well plate 12 in any conventional manner so as to define a sealed interface between outer peripheral portion 118 of upper surface 116 of lid plate 100 and lower surface 26 of well plate 12.

With outer peripheral portion 118 of upper surface 116 of lid plate 100 bonded to lower surface 26 of well plate 12, inner portion 120 of upper surface 116 of lid plate 100 is directed towards and partially defines media cavity 60. In addition, it is intended for inner portion 120 of upper surface 116 of lid plate 100 to be generally parallel to and spaced from terminal faces 86 of the plurality of voxels 76. In other words, the distance D1 between inner portion 120 of upper surface 116 of lid plate 100 and upper face 72 is greater than the distance D2 between terminal faces 86 of the plurality of voxels 76 and upper face 72, FIG. 8.

Referring to FIGS. 11-13, device 10 further includes sample transport device 124 fabricated from a plastic material such as polyvinylchloride, polystyrene or polypropylene. Sample transport device 124 includes sample plate 122 having a generally rectangular configuration corresponding in size and shape to well plate 12. However, other configurations are possible without deviating from the scope of the present invention. Sample plate 122 is defined by first and second generally parallel ends 126 and 128, respectively, interconnected first and second generally parallel sides 130 and 132, respectively, perpendicular thereto. Sample plate 122 further includes upper surface 134 and lower surface 136.

A plurality of capillary tubes, designated collectively by the reference numeral 138, depend from lower surface 136 of sample plate 122. It is intended for the number of capillary tubes 138 depending from lower surface 136 of sample plate 122 to correspond to the number of wells 14 in well plate 12. For example, in the depicted embodiment, nine (9) capillary tubes 138 depend from lower surface 136 of sample plate 122. Capillary tubes 138 are arranged in three (3) rows and three (3) columns corresponding the three (3) rows and three (3) columns of wells 14 in well plate 12. It is further intended that capillary tubes 138 are arranged so as to be insertable simultaneously into a corresponding well 14 in well plate 12, for reasons hereinafter described. It can be understood that the number and arrangement of capillary tubes 138 depending from lower surface 136 of sample plate 122 may be varied, without deviating from the scope of the present invention.

Each capillary tube 138 is defined by an outer surface 140 intersecting lower surface 136 of sample plate 122 at intersection 142 and a lower edge 144 spaced from lower surface 136 of sample plate 122 and defining the outer periphery of terminal surface 145 of each capillary tube 138. In the depicted embodiment, outer surfaces 140 have a generally rectangular configuration. However, other configurations are possible without deviating from the scope of the present invention. Each capillary tube 138 further includes an inner surface 146 defining passage 148 therethrough. In the depicted embodiment, inner surfaces 146 have a generally rectangular configuration. However, other configurations are possible without deviating from the scope of the present invention. The upper ends of inner surfaces 146 of capillary tubes 138 intersect upper surface 134 of sample plate 122 at intersections 150 which, in turn, define corresponding upper openings 152 in sample plate 122. Each upper opening 152 in sample plate 122 communicates with passage 148 through a corresponding capillary tube 138. Similarly, the lower ends of inner surfaces 146 of capillary tubes 138 intersect terminal surfaces 145 of corresponding capillary tubes 138 at intersections 154 which, in turn, define corresponding lower openings 156 in the corresponding capillary tubes 138. Lower opening 156 in terminal surface 145 of capillary tube 138 communicates with passage 148 through capillary tube 138.

Referring to FIGS. 14-16, device 10 may further include indicia plate 160 receiveable on upper surface 24 of well plate 12. Indicia plate 160 has a generally rectangular configuration and corresponds in size and shape to well plate 12. However, other configurations are possible without deviating from the scope of the present invention. Indicia plate 160 is defined by first and second generally parallel ends 162 and 164, respectively, interconnected first and second generally parallel sides 166 and 168, respectively, perpendicular thereto. Indicia plate 160 further includes upper surface 169 and lower surface 171.

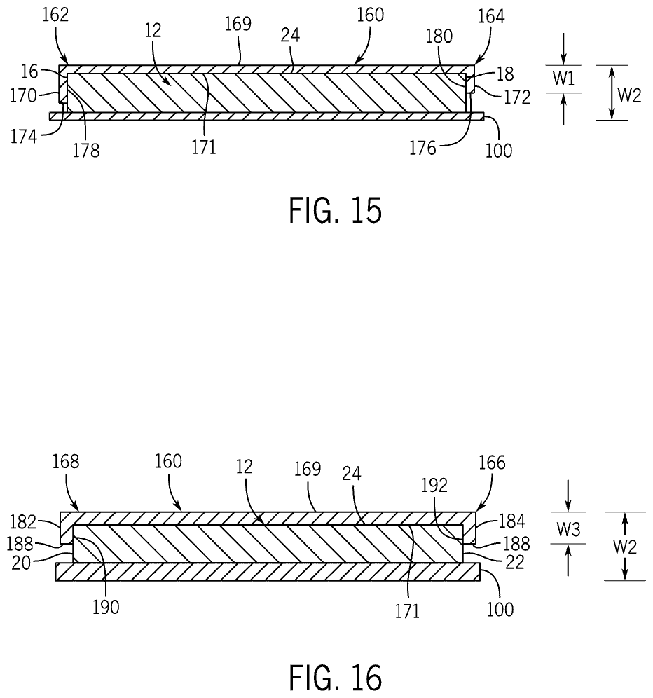

As best seen in FIG. 15, first and second alignment walls 170 and 172, respectively, depend from first and second ends 162 and 164, respectively, and terminate at terminal ends 174 and 176, respectively. It is contemplated for first and second alignment walls 170 and 172, respectively, to have a width W1 less than width W2 of the combination of well plate 12 and lid plate 100 bonded thereto. First and second alignment walls 170 and 172, respectively, further include inner surfaces 178 and 180 directed towards each other and engageable with corresponding first and second ends 16 and 18, respectively, of well plate 12.

Similarly, as best seen in FIG. 16, third and fourth alignment walls 182 and 184, respectively, depend from first and second sides 166 and 168, respectively, and terminate at terminal ends 186 and 188, respectively. It is contemplated for third and fourth alignment walls 182 and 184, respectively, to have a width W3 which is less than width W2 of the combination of well plate 12 and lid plate 100. Third and fourth alignment walls 182 and 184, respectively, further include inner surfaces 190 and 192 directed towards each other and engageable with corresponding first and second sidewalls 20 and 22, respectively, of well plate 12.

A plurality of openings, designated collectively by the reference numeral 194, extend through indicia plate 160 between upper surface 169 and lower surface 171 thereof. It is intended for each opening 194 to be aligned with and overlap either a single well 14 or a selected grouping of wells, generally designated by the reference numeral 196, in well plate 12 with indicia plate 160 received on upper surface 24 of well plate 12, as hereinafter described. It the depicted embodiment, openings 194 through indicia plate 160 are arranged in four (4) rows 198a-198d and eight (8) columns 200a-200h. However, it can be understood that the number and arrangement of openings 194 through indicia plate 160 may be varied depending on the location and distribution of wells in upper surface 24 of well plate 12 is identical in structure, without deviating from the scope of the present invention.

Indicia plate 160 includes indicia identifying rows 198a-198d, e.g. numbers 1-4, on upper surface 196 thereof at a location adjacent corresponding openings 178 in first column 200a. Similarly, indicia plate 160 includes indicia identifying columns 200a-200h, e.g. numbers 1-8, on upper surface 196 thereof at a location adjacent first row 198a. The indicia allow a user to simply and easily identify a row and/or column corresponding to a desired well 14 or a selected grouping of wells 14 in well plate 12.

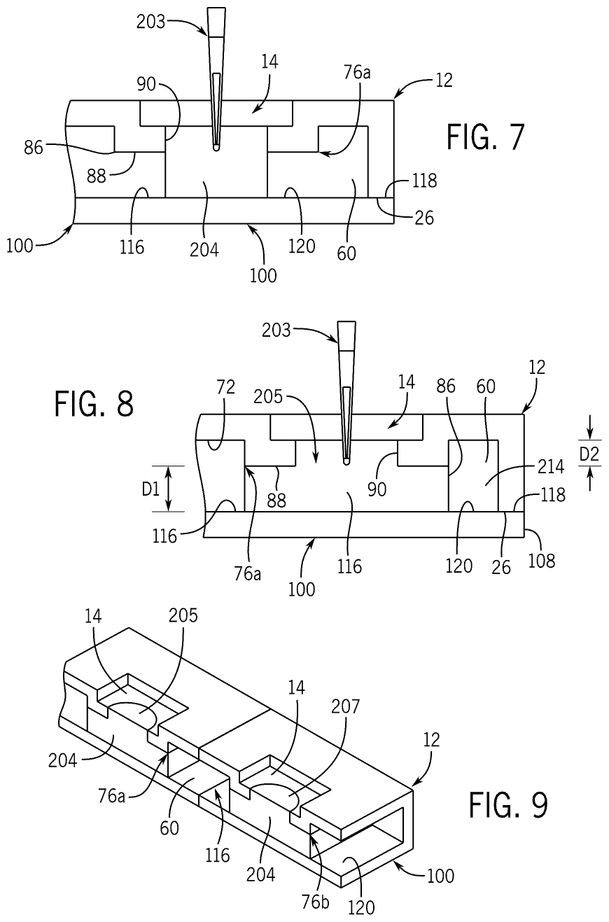

Referring to FIGS. 7-9, in operation, a user deposits a desired hydrogel 204 loaded with user selected cells, e.g. tumor cells, stromal cells or immune cells, onto terminal face 88 of a desired voxel 76 and into media cavity 60. More specifically, micropipette 203 is inserted into well 14 in axial alignment with a desired voxel 76, e.g. first voxel 76a. Thereafter, hydrogel 204 is pipetted from micropipette 203 into passage 90 and onto terminal face 88 of first voxel 76a. As hydrogel 204 enters media cavity 60, hydrogel 204 flows radially outward from passage 90 along terminal face 88 of voxel 76a. As hydrogel 204 flows radially outward along terminal face 88 of first voxel 76a, hydrogel 204 gets pinned between pinning edge 86 of voxel 76a and upper surface 116 of lid plate 100, FIG. 8. Thereafter, hydrogel 204 is polymerized and forms a first cell structure 205.

In the same manner, the same or a different hydrogel 204 loaded with user selected cells, e.g. tumor cells, stromal cells or immune cells, is pipetted into passage 90 and onto terminal face 88 of a desired voxel 76, e.g. second voxel 76b, FIG. 9. Again, as hydrogel 204 enters media cavity 60, hydrogel 204 flows radially outward from passage 90 along terminal face 88 of second voxel 76b. As hydrogel 204 flows radially outward along terminal face 88 of second voxel 76b, hydrogel 204 gets pinned between pinning edge 86 of second voxel 76b and upper surface 116 of lid plate 100. Hydrogel 204 is polymerized and forms a second cell structure 207. With first and second cell structures 205 and 207, respectively, pinned at corresponding pinning edges 86 of first and second voxels 76a and 76b, respectively, first and second cell structures 205 and 207, respectively, are spaced from each other, thereby allowing for fluid flow thereabout, for reasons hereinafter described.

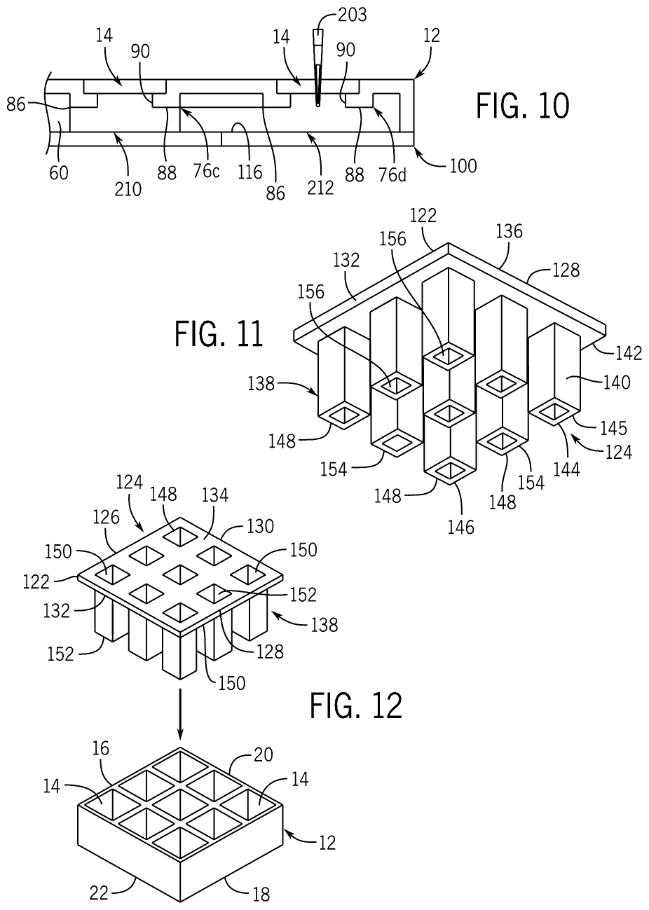

Alternatively, it can be understood that a user may construct a cell structure, e. g third cell structure 210, in contact with an adjacent cell structure, e.g. fourth cell structure 212, if desired. More specifically, referring to FIG. 10, third cell structure 210 may be formed on a desired voxel 76, e.g. third voxel 76c. Thereafter, micropipette 203 is inserted into well 14 in axial alignment with a desired voxel 76, e.g. fourth voxel 76d adjacent to third cell structure 210. Hydrogel 204 is pipetted from micropipette 203 into passage 90 and onto terminal face 88 of fourth voxel 76d. As hydrogel 204 enters media cavity 60, hydrogel 204 flows radially outward from passage 90 along terminal face 88 of fourth voxel 76d. As hydrogel 204 flows radially outward along terminal face 88 of fourth voxel 76d, hydrogel 204 gets pinned between pinning edge 86 of fourth voxel 76d and upper surface 116 of lid plate 100. The user may continue to pipette hydrogel 204 into passage 90 and onto terminal face 88 of fourth voxel 76d to overcome the pinning of hydrogel 204 between pinning edge 86 of fourth voxel 76d and upper surface 116 of lid plate 100 such that hydrogel 204 continues to flow radially outward from passage 90 along terminal face 88 of fourth voxel 76d and into media cavity 60 until hydrogel 204 engages third cell structure 210. Hydrogel 204 is them polymerized to form fourth cell structure 212 in contact with third cell structure 210. The processes heretofore described may be repeated such that a desired pattern of cells structures are provided in media cavity 60.

It can be appreciated that by pinning the cell structures at pinning edges 86 of voxels 76, the portion of media cavity 60 surrounding the cell structures remains empty. As such, growth media 210, FIG. 8, may be pipetted into media cavity 60 through passages 90 not in communication with corresponding cell structures. In addition, each of the wells 14 are filled with growth media 214, such that cell structures, e.g. first and second cell structures 205 and 207, respectively, are surrounded by growth media 214. Once wells 14 are filled with growth media 210, a user may deposit nutrients, metabolites, drugs or the like in selected wells 14. The nutrients, metabolites, drugs or the like flow into media cavity 60 and are delivered to the cells loaded in cell structures, e.g. first and second cell structures 205 and 207, respectively.

It can be understood that the cells loaded in cell structures, e.g. first and second cell structures 205 and 207, respectively, in media cavity 60 maintain their spatial information while the nutrients, metabolites, drugs or the like are delivered thereto. As such, the cells loaded in cell structures, e.g. first and second cell structures 205 and 207, respectively, in media cavity 60 may be observed and studied in a spatial manner with destruction of the environment. By way of example, first and second cell structures 205 and 207, respectively, may be observed in situ within device 10 though lid plate 100 by means of an imaging device or the like (not shown).

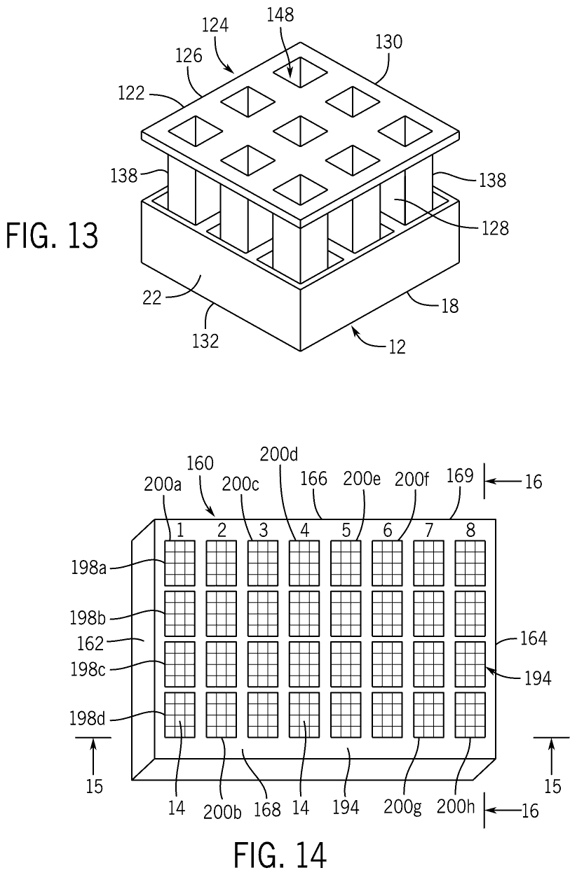

In addition, samples of fluid in wells 14 may be drawn for further analysis and study, e.g. by means of sample transport device 124. More specifically, sample transport device 124 may be positioned such that capillary tubes 138 are inserted into a corresponding wells 14 in well plate 12, FIGS. 12-13. Capillary action draws small aliquots of fluid from wells 14 into corresponding capillary tubes 138. Sample transport device 124 may be repositioned such that capillary tubes 138 having the small aliquots of fluid from wells 14 therein are removed from corresponding wells 14 in well plate 12 and transported to a desired location to allow for further analysis of the small aliquots of fluid in capillary tubes 138.

Although the best mode contemplated by the inventors of carrying out the present invention is disclosed above, practice of the above invention is not limited thereto. It will be manifest that various additions, modifications and rearrangements of the features of the present invention may be made without deviating from the spirit and the scope of the underlying inventive concept.

We claim:

1. A cellular arrangement and microfluidic patterning device, comprising:
a plate including:
a first face having a well formed therein, the well partially defined by a bottom surface spaced from the first face;
a cavity partially defined by a first surface directed away from the well and a second surface spaced from the first surface and directed towards the well;
a voxel projecting from the first surface into the cavity and terminating at a terminal surface spaced from the second surface, the voxel being axially aligned with the well; and
an aperture extending between the bottom surface of the well and the terminal surface of the voxel.

2. The cellular arrangement and microfluidic patterning device of claim 1 wherein the terminal surface of the voxel is defined by a generally rectangular peripheral edge.

3. The cellular arrangement and microfluidic patterning device of claim 1 wherein:
the first surface is spaced from the second surface by a distance; and
the terminal surface of the voxel is spaced from the second surface by a distance which is less than the distance between the first and second surfaces.

4. The cellular arrangement and microfluidic patterning device of claim 1 wherein the well is a first well and the voxel is a first voxel and wherein the plate includes:
a second well formed in the first face, the second well partially defined by a bottom surface spaced from the first face;
a second voxel projecting from the first surface into the cavity and terminating at a terminal surface spaced from the second surface, the second voxel being axially aligned with the second well; and
an aperture extending between the bottom surface of the second well and the terminal surface of the second voxel.

5. The cellular arrangement and microfluidic patterning device of claim 4 wherein the terminal surface of the second voxel is defined by a generally rectangular peripheral edge.

6. The cellular arrangement and microfluidic patterning device of claim 4 wherein the terminal surfaces of the first and second voxels lie in a common plane.

7. The cellular arrangement and microfluidic patterning device of claim 4 wherein the first and second voxels are spaced from each other along the first surface by a distance.

8. The cellular arrangement and microfluidic patterning device of claim 4 further comprising a media transfer tool, the media transfer tool including:
a panel; and
first and second tubular projections extending from the panel and having terminal ends, the terminal ends of the projections configured to be received in corresponding first and second wells of the plate.

9. The cellular arrangement and microfluidic patterning device of claim 4 further comprising a guide receivable on the first face of the plate, the guide including:
a panel having:
first and second faces and first and second apertures therethrough that are alignable with corresponding first and second wells in the plate; and
indicia provided on the first face to facilitate identification of locations of first and second wells in the plate.

10. A cellular arrangement and microfluidic patterning device, comprising:
a plate including:
a generally planar face;
an array of wells providing in the face, each well of the array of wells being partially defined by a bottom surface spaced from the face;
a cavity partially defined by a first surface directed away from the array of wells and a second surface spaced from the first surface and directed towards the array of wells;
a plurality of voxels projecting from the first surface into the cavity, each voxel of the plurality of voxel terminating at a terminal surface spaced from the second surface and being axially aligned with a corresponding well of the array of well; and
a plurality of apertures, each aperture extending between the bottom surface of one of the wells of the array of wells and the terminal surface of a corresponding voxel of the plurality of voxels.

11. The cellular arrangement and microfluidic patterning device of claim 10 wherein each terminal surface of the plurality of voxels is defined by a generally rectangular peripheral edge.

12. The cellular arrangement and microfluidic patterning device of claim 10 wherein at least one voxel of the plurality of voxels has a rectangular cross-section.

13. The cellular arrangement and microfluidic patterning device of claim 10 wherein the terminal surfaces of the plurality of voxels lie in a common plane.

14. The cellular arrangement and microfluidic patterning device of claim 10 wherein:
the first surface is spaced from the second surface by a distance; and
the terminal surfaces of the plurality of voxels are spaced from the second surface by a distance which is less than the distance between the first and second surfaces.

15. The cellular arrangement and microfluidic patterning device of claim 10 wherein each voxel of the plurality of voxels have identical configurations.

16. The cellular arrangement and microfluidic patterning device of claim 10 wherein each voxel of the plurality of voxels is spaced from an adjacent voxel of the plurality of voxels by a distance.

17. The cellular arrangement and microfluidic patterning device of claim 10 further comprising a media transfer tool, the media transfer tool including:
a panel; and
an array of tubular projections extending from the panel and having terminal ends, the terminal ends of the projections are receivable in a corresponding well of the array of wells of the plate.

18. The cellular arrangement and microfluidic patterning device of claim 10 wherein the array of wells are arranged in rows and columns in the face of the panel.

19. The cellular arrangement and microfluidic patterning device of claim 18 further comprising a guide receivable on the first face of the plate, the guide including:
a panel having:
first and second faces and an array of apertures therebetween, each aperture of the array of apertures being alignable with corresponding well of the array of wells; and
first and second sets of indicia along the first face of the panel, the first set of indicia extending along a first axis and identify each row of wells of the array of wells and the second set of indicia extending along a second axis and identify each column of wells of the array of wells.

20. A method of studying the positional context of cells, comprising the steps of:
providing an array of wells in a face of a plate, the plate including:
a cavity communicating with each of the wells of the array of wells; and
a plurality of voxels projecting into the cavity, each voxel aligned with a corresponding well and from a first surface into the cavity, each voxel of the plurality of voxel terminating at a terminal surface;
depositing three-dimensional hydrogel figures on at least a portion of the terminal surfaces of the plurality of voxels; and
depositing media in the cavity such that the media surrounds the hydrogel figures.

21. The method of claim 20 wherein each hydrogel figure includes cells suspended therein.

22. The method of claim 20 comprising the additional step of arranging the arrays of wells in rows and columns.

23. The method of claim 21 comprising the additional step of providing indicia on the face of the plate to identify the rows and columns of the array of wells.

24. The method of claim 20 comprising the additional step of pinning the hydrogel figures deposited on the at least the portion of the terminal surfaces of the plurality of voxels.

25. The method of claim 20 further comprising the steps of:
filling the array of wells with the media; and
selectively drawing the media out of the array of wells with a media transfer tool.

26. The method of claim 25 wherein the media transfer tool includes:
a panel; and
an array of tubular projections extending from the panel and having terminal ends, the terminal ends of the projections are receivable in corresponding wells of the array of wells of the plate.
